(19) United States
(12) Patent Application Publication
(10) Pub. No.: US 2011/0245864 A1
(43) Pub. Date: Oct. 6, 2011

(54) Medical Instrument

(76) Inventors: Regis Besse, Guyancourt (FR); Yann Thouement, Les essarts le roi (FR)

(21) Appl. No.: 13/077,432

(22) Filed: Mar. 31, 2011

(30) Foreign Application Priority Data
Apr. 1, 2010 (DE) 10 2010 013 916.5

(51) Int. Cl. A61B 17/28

(52) U.S. Cl. 606/205

(57) ABSTRACT

A medical instrument with a hollow shaft having a handle including at least two gripping members positioned on its proximal end and a tool positioned on its distal end, so that the tool may be actuated by the handle via an actuating element that is mounted so that it can slide axially in the shaft. A medical instrument that ensures simple structure and a direct and precise control of the tool having gripping members of the handle and the actuating element being actively interconnected by a stud-slit control device.

### CROSS-REFERENCE TO RELATED APPLICATIONS

The present application claims priority of German patent application No. 10 2010 013 916.5 filed on Apr. 1, 2010.

### FIELD OF THE INVENTION

The invention relates to a medical instrument with a hollow shaft having a handle including at least two gripping members positioned on its proximal end and a tool positioned on its distal end, so that said tool can be actuated by means of the handle via an actuating element that is mounted so that it can slide axially in the shaft.

### BACKGROUND OF THE INVENTION

A generic medical instrument is known, for example, from U.S. Pat. No. 5,501,698 A. In this known medical instrument the handle and actuating element are coupled to one another by articulated links that are pivotally mounted on the gripping members as well as on the actuating element. Use of the interposed pivotable articulated links, because of the many components that are to be coupled to one another, entails a high installation cost.

Patent DE 693 25 625 T2 describes an instrument to apply a surgical clamp in body tissue and/or to apply a surgical clamp to connect blood vessels. The generic instrument comprises an actuating mechanism with a cam groove-pin connection. The deformation cam, in which the cam groove is positioned, is of flat configuration.

On this basis, it is the object of the invention to provide a medical instrument of the aforementioned type that is of simple structure and ensures a direct and precise control of the tool.

This object is achieved by a medical instrument described below. It is particularly important here that the actuating element should comprise a tension-compression element that is configured as a cross-shaped element that is mounted to slide axially in the shaft and having a base body aligned in the instrument's longitudinal axis and at least two rod-type arms extending outward, and that the gripping members of the handle and the arms of the tension-compression element should be actively interconnected by means of a stud-slit control device.

### SUMMARY OF THE INVENTION

The aforementioned cross-shaped element is understood to be a base body with at least two arms extending outward. The arms can be positioned either at right angles or at an obtuse or acute angle to the base body, so that the cross-shaped element can be configured as T-shaped, Y-shaped, or arrow-shaped, with a projection from the base body on both sides. In a preferred embodiment, the extending arms are positioned in particular in mirror symmetry to the base body and meet in a common point on the longitudinal axis of the base body. This is of great advantage especially in static and constructive terms. The production process of the base body can also be simplified thanks to a mirror-symmetrical arrangement.

A rod-shaped configuration of the arms extending from the base body allows a solution that is both economical in terms of space and also stable. “Rod-shaped” is taken in this context to indicate an element whose cross-section shape, in particular, can be of either round or square configuration and in which the ratio between rod length and rod cross-section dimension is at least 3:2 or in particular 3:1 or greater.

Configuration of the coupling between the handle and the actuating element mounted in the shaft as a stud-slit control device offers the possibility of a direct transmission of the pivotal motion of the gripping members of the handle into the axial movement of the actuating element without interposing any further redirecting elements.

For the configuration of the stud-slit control device, an embodiment of the invention involves said stud-slid device having guide tracks directly or indirectly configured on one of the components that are to be coupled together, namely the gripping members or the arms, and control studs that are configured directly or indirectly on the other of the components that are to be coupled together, namely the arms or gripping members, said control studs being mounted in the guide tracks.

According to another embodiment of the invention, the guide tracks are configured in the gripping members and the control studs are coupled with the arms. This type of configuration of the stud-slit control device is an embodiment of the invention that is especially simple to produce and easy to assemble.

Another embodiment of the invention involves the actuating element being configured in multiple parts in the instrument longitudinal axis, so that the individual parts of the actuating element can be coupled together via a coupling mechanism in order to allow the assembly of the stud-slid control device independently of the actuating element mounted in the shaft.

According to another embodiment of the invention, the coupling mechanism is equipped with a holding fixture to couple connecting elements of different cross-section shape. The connecting element links the tension-compression element with the tool on the distal end of the actuating element and transmits the axial movement of the actuating element, which involves in particular of the tension-compression element, the coupling mechanism and the connecting element, to the tool. This makes possible a modular instrument concept because connecting elements with different cross-section shape, in particular cross-section size and cross-section form, can be used in the holding fixture of a single handgrip. In this manner the user, with one handgrip, can actuate numerous connecting elements, for example sets of forceps with different diameter and tool configuration, and the number of instruments on the operating table can be reduced.

In another embodiment, the holding fixture of the coupling mechanism comprises a locking element that safeguards the proximal end of a connecting element, which can vary in its cross-section shape, from accidental release in the holding fixture. Thereby the connecting element is no longer able to slide in the axial direction by means of the locking element of the holding fixture independently of the compression-tension element, and thus a reliable force transmission is obtained from the gripping members via the actuating element with the connecting element and the cross-shaped compression-tension element onto the tool.

The control studs are advantageously configured on a compression-tension element that forms the proximal end of the actuating element.

Alternatively it is also possible of course to position the control studs directly on the actuating element.

According to another embodiment of the invention, the compression-tension element may be configured as a cross-shaped stem mounted in the shaft with a base body aligned in the instrument longitudinal axis and two arms extending outward, so that the control studs are positioned at the free ends of the two arms. Additionally, according to another embodiment, the cross-shaped compression-tension element may be configured as a T-shaped stem with a base body aligned in the instrument longitudinal axis and two arms essentially extending rectilinearly outward, and with the control studs positioned on the free ends of the two arms.

To be able to continually return the tool as well as the gripping members of the handle automatically back into a starting position, another embodiment of the invention involves that the actuating element may be pretensed in distal direction by a spring element, preferably a pressure spring.

In another embodiment of the invention, the control studs may be mounted to be pivotable around their longitudinal axis, and/or that rotatable casters should be positioned around stationary control studs. Rotatable mounting of the control studs reduces the friction resistance in moving the control studs inside the guide tracks.

Further properties and advantages of the invention can be seen from the appended drawings, in which an embodiment of an inventive medical instrument is presented only by way of example, without restricting the invention to said embodiment.

### BRIEF DESCRIPTION OF THE DRAWINGS

FIG. 1 shows a schematic side view of an inventive medical instrument;

FIG. 2 shows an enlarged view of detail II from FIG. 1;

FIG. 3 shows a longitudinal section through the detail seen in

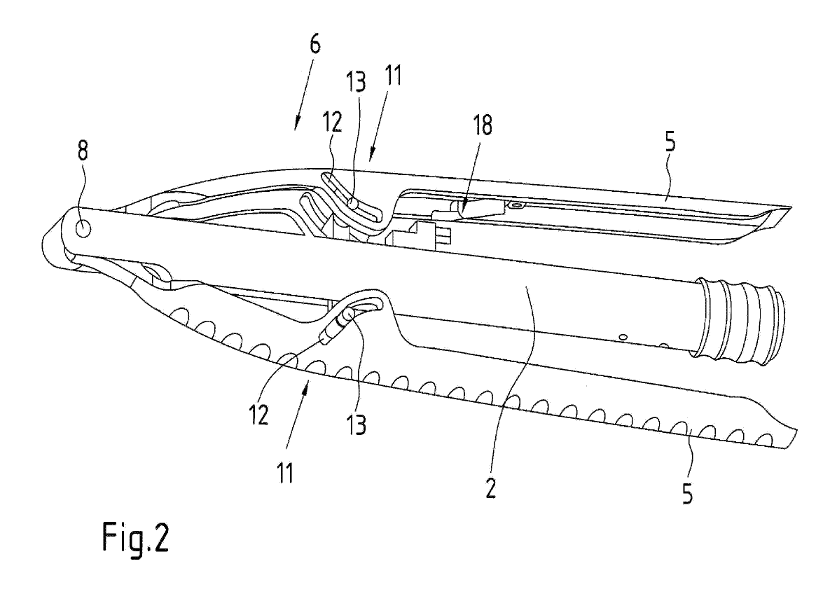

FIG. 2;

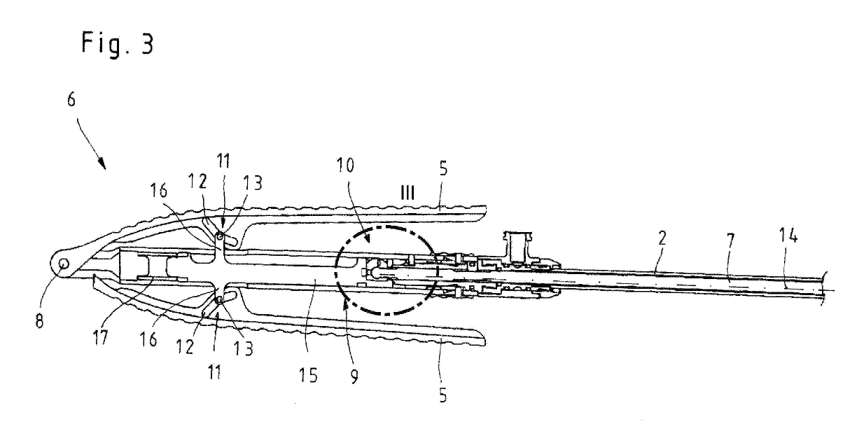

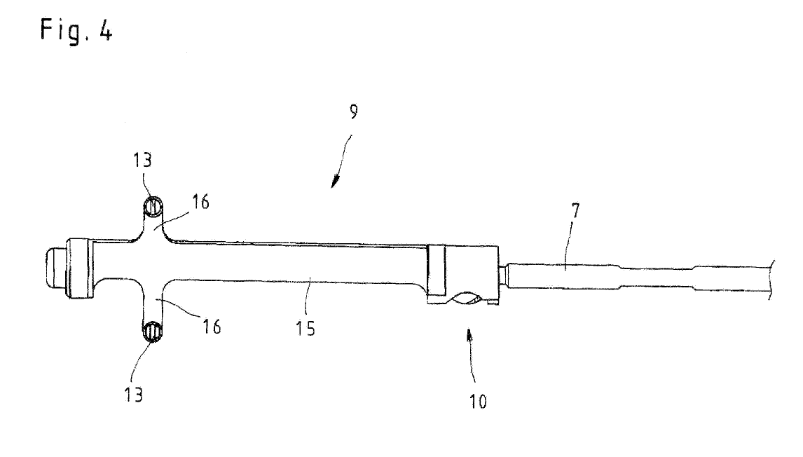

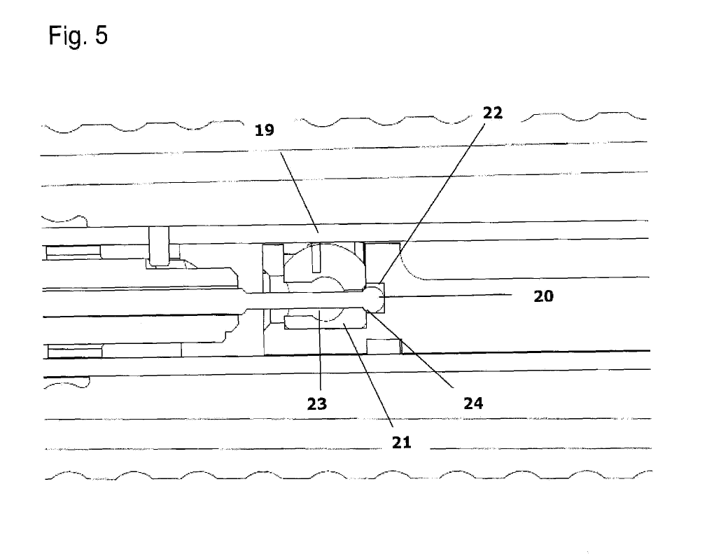

FIG. 4 shows a view of the compression-tension element according to FIG. 3; and

FIG. 5 shows an enlarged view of detail III from FIG. 3.

### DETAILED DESCRIPTION OF THE INVENTION

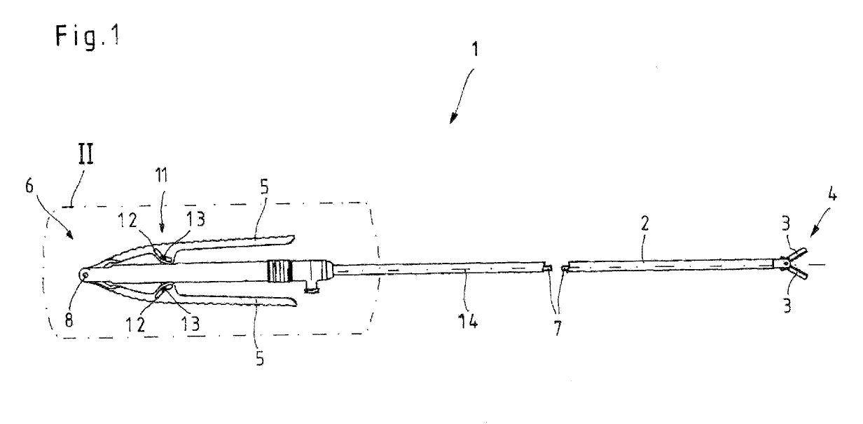

The medical instrument 1 shown in FIG. 1 includes a hollow shaft 2 that has a tool 4 having two jaw members 3 positioned on its distal end and a handle 6 having two gripping members 5 positioned on its proximal end. In the illustrated embodiment, the tool 4 has two jaw members 3 that can pivot relative to one another. To convert the jaw members 3 of the tool 4 from an open starting position into a closed working position, the pivotable jaw members 3 of the tool 4 and the gripping members 5 of the handle 6 are actively linked together by an actuating element 7 mounted so that it can slide axially in the hollow shaft 2.

Alternatively to the illustrated configuration of the tool 4 with two pivotable jaw members 3, it is also possible to configure the tool 4 so that it has a rigid jaw member 3 and only one pivotable jaw member 3. It is likewise possible to configure the tool 4 to have only one tool member, for example as a hook or point that can be moved forward and back by the handle 6 or the actuating element 7.

The gripping members 5 of the handle 6 in the illustrated embodiment are mounted on the proximal end of the shaft 2 to pivot around a common pivot axis 8 and with their free ends point in the direction of the distal end of the shaft 2.

In the embodiment illustrated in FIGS. 3 and 4, the actuating element 7 is configured in two parts in such a way that the proximal end of the actuating element 7 is configured as a separate compression-tensions element 9 that can be coupled essentially without free play with the distal part of the actuating element 7 by a coupling mechanism 10.

As can be seen from FIG. 5, the coupling mechanism 10 has a holding fixture 19 with a locking element 21, into which the connecting element 20 is inserted. The connecting element 20 is configured as a sphere on its proximal end. When the gripping members 5 are completely opened, the cross-shaped element, the base body 15 and the arms 16 are in the distal position. The locking element 21 accordingly is found in a position that makes possible the insertion of a connecting element 20. Because the spherical element on the proximal end of the connecting element 20 has a smaller diameter than a borehole 24 in the locking element 21, it is accordingly guided through the locking element 21 and is inserted in the smaller holding fixture 22 of the two holding fixtures. If then the gripping members 5 are pressed together in the direction of the base body 15, the cross-shaped compression-tension element 9 moves in the proximal direction. In the process, the locking element 21 is rotated into a locking position by control pins (not visible in FIG. 5). Thereby the connecting element 20 is firmly anchored outside the locking element 21 in the small holding fixture 22 and can no longer be slid axially. The cross-shaped compression-tension element 9 is now firmly connected with the connecting element 20 by the holding fixture 19.

In another embodiment, the spherical-shaped element on the proximal end of the connecting element 20 may have a diameter greater than the borehole 24, so that it cannot be passed through the locking element 21 and is held in a large holding fixture 23 in the center of the locking element 20. By actuating the gripping members 5, the locking element 21 is rotated into a locking position by control pins in this application as well, so that the proximal end of the connecting element 20 can no longer release itself from the center of the locking element 21 and the compression-tension element is firmly coupled with the connecting element 20.

A correlation of the connecting element 20 to an appropriate holding fixture corresponding to its cross-section shape is thereby assured.

In another embodiment, the actuation element 7 may be configured as consisting of a single part or else of more than two parts, although in the multi-part configuration all parts of the actuating element 7 may be coupled to one another by corresponding coupling mechanisms 10.

The structure of the compression-tension element 9 as well as the type of conversion of the pivotal movement of the gripping members 5 of the handle 6 into the exclusive axial movement of the actuating element 7 can be seen in particular from FIG. 3.

As shown in FIG. 3, the gripping members 5 of the handle 6 and the actuating element 7 are actively interconnected by a stud-slit control device 11. In the illustrated embodiment, the stud-slit control device 11 has guide tracks 12 configured on the gripping members 5 of the handle 6 and control studs 13 configured on the actuating element 7 and mounted in the guide tracks 12. The control studs 13 here are positioned on the proximal part of the actuating element 7 that is configured as a separate compression-tension element 9.

In another embodiment, the control studs 13 may be arranged on a one-piece actuation element or arranged on the gripping members 5 and to configure the corresponding guide tracks on the actuation element 7.

To reduce friction resistance between the control studs 13 and the guide tracks 12 in moving the control studs 13 inside the guide tracks 12, the control studs 13 may be mounted so that they can be rotated around their longitudinal axis so that said studs roll in the guide tracks 12 when the gripping parts 5 are actuated. In another embodiment, rotatable rollers may also be applied on stationary studs.

The compression-tension element 9 that forms the proximal part of the actuation element 7 is configured in the illustrated embodiment as a T-shaped stem mounted so that it can slide axially in the shaft 2 with a base body 15 aligned in the instrument longitudinal axis 14 and two rod-shaped arms 16 extending outward essentially in rectilinear manner and in mirror symmetry, whereby the control studs 13 are positioned on the free ends of the two rod-shaped arms 16.

The pivotal movement of the gripping members 5 of the handle 6 is converted into a purely axial movement of the actuation element 7 in the longitudinal direction of the shaft 2 by the control studs 13 of the stud-slit control device 11 that are mounted in the guide tracks 12.

In the illustrated embodiment of the stud-slit control device 11, the guide tracks 12 are geometrically configured in such a way that, upon pulling apart the gripping members 5 of the handle 6, the compression-tension element 9 as well as the actuating element 7 coupled with the compression-tension element 9 is pushed distally in the axial direction and the pivotable jaw members 3 of the tool 4 move into the open position.

Conversely, upon pressing together the gripping members 5 of the handle 6, the compression-tension element 9 as well as the actuating element 7 coupled with the compression-tension element 9 is pulled proximally in the axial direction, so that the pivotable jaw members 3 of the tool 4 are moved into the closed position.

As can be further seen from FIG. 3, the actuating element 7 is pretensed in the distal direction by a spring element 17, which is supported on the proximal side on the proximal end of the shaft 2 and is contiguous on the distal side with the proximal end of the compression-tension element 9. By means of this spring element 17, which is preferably configured as a pressure spring, the compression-tension element 9 and the actuating element 7 are pressed in the distal direction so that the pivotable jaw members 3 of the tool 4 are converted into the open position and the gripping members 5 of the handle 6 are converted into the spread-apart position by means of the control studs 13 mounted in the guide tracks of the gripping members 5.

To free up the operator, the gripping members 5 of the handle 6 as shown in FIG. 2 may be fixed in the pressed-together position, that is, with closed jaw members 3 of the tool 4, by means of a blocking mechanism 18, so that the operator is not obliged to constantly press the gripping members 5 of the handle 6 together by manual force.

A medical instrument 1 of this configuration is characterized by the configuration of the coupling between the gripping members 5 of the handle 6 and the actuating element 7 as a stud-slit control device 11, thereby ensuring simplicity of structure as well as a control of the jaw members 3 of the tool 4 that is direct, precise and essentially free of play.

1-12. (canceled)

13. A medical instrument comprising:
a hollow shaft having a handle including at least two gripping members positioned on its proximal end, and a tool positioned on its distal end, so that said tool can be actuated by means of the handle via an actuating element that is mounted so that it can slide axially in the shaft,
wherein the actuating element includes a compression-tension element that is configured as a cross-shaped element that is mounted so that it can slide axially in the shaft, with a base body aligned in the longitudinal axis of the medical instrument and at least two rod-shaped arms extending outward, and
wherein the gripping members of the handle and the arms of the compression-tension element are actively linked together by a stud-slit control device.

14. The medical instrument of claim 13, wherein the rod-shaped arms are positioned and configured so that they are mirror-symmetrical on the base body.

15. The medical instrument of claim 13, wherein the stud-slit control device has guide tracks configured directly or indirectly on one of the gripping members or the arms, the gripping members and the arms being coupled together, and control studs that are configured directly or indirectly on the other of the gripping members or the arms, said control studs being mounted in the guide tracks.

16. The medical instrument of claim 15, wherein the guide tracks are configured in the gripping members of the handle and the control studs are coupled with the arms.

17. The medical instrument of claim 15, wherein the control studs are positioned on the free ends of the two arms.

18. The medical instrument of claim 15, wherein the base body is aligned in the longitudinal axis of the instruction and two essentially rectilinear arms extending outward are in a T-shaped configuration, whereby the control studs are positioned on the free ends of the two arms.

19. The medical instrument of claim 13, wherein the actuating element is in the longitudinal axis of the instrument and is configured in several parts, so that the individual parts of the actuating element can be coupled together by at least one coupling mechanism.

20. The medical instrument of claim 19, wherein the coupling mechanism has a holding fixture with at least one holding part for coupling connecting elements of different cross-section shape.

21. The medical instrument of claim 20, wherein the holding fixture has a locking element for coupling at least one connecting element.

22. The medical instrument of claim 19, wherein the holding fixture has holding parts of different cross-section shape for holding connecting elements of corresponding cross-section shape.

23. The medical instrument of claim 13, wherein the compression-tension element forms the proximal end of the actuating element.

24. The medical instrument of claim 13, wherein the actuating element is pretensed in the distal direction by a spring element, preferably a pressure spring.

25. The medical instrument of claim 14, wherein the control studs are mounted so that they can rotate around their longitudinal axis.
